(19) United States
(12) Patent Application Publication
(10) Pub. No.: US 2003/0204861 A1
(43) Pub. Date: Oct. 30, 2003

(54) Transgenic animal model for spliceosome-mediated RNA trans-splicing

(76) Inventors: Madaiah Puttaraju, Germantown, MD (US); Lloyd G. Mitchell, Bethesda, MD (US); John F. Engelhardt, Iowa City, IA (US); Xiaoming Liu, Iowa City, IA (US)

Correspondence Address: BAKER & BOTTS, 30 ROCKEFELLER PLAZA, NEW YORK, NY, 10112

(21) Appl. No.: 10/136,723

(22) Filed: Apr. 30, 2002

(51) Int. Cl. A01K 67/027

(52) U.S. Cl. 800/18

(57) ABSTRACT

The present invention relates to development of an animal model system for in vivo testing of spliceosome-mediated RNA trans-splicing reactions. The present invention provides transgenic animals, and methods for generating such animals, that have been genetically engineered to expresses a target precursor messenger RNA molecule (target pre-mRNA) that serves as a substrate for a trans-splicing reaction. Specifically, the transgenic animals contain at least one transgene capable of expressing a target pre-mRNA molecule. The invention provides methods, based on utilization of the transgenic animals, for assessing the specificity and efficiency of a pre-trans-splicing molecule (PTM) designed to interact with a target pre-mRNA and mediate a trans-splicing reaction resulting in the generation of a novel chimeric RNA molecule. The present invention further relates to the transgenic expression of PTM molecules in animals to determine gene function, i.e, functional genetics. The present invention is based on the successful generation of a transgenic animal expressing a target pre-mRNA and, moreover, the use of that animal to detect accurate in vivo trans-splicing reactions in the presence of a PTM.

The present invention involves subject matter developed under NIH Grant No. 2R44DK56526.

### 1. INTRODUCTION

The present invention relates to development of an animal model system for in vivo testing of spliceosome-mediated RNA trans-splicing reactions. The present invention provides transgenic animals, and methods for generating such animals, that have been genetically engineered to express a target precursor messenger RNA molecule (target pre-mRNA) that serves as a substrate for a trans-splicing reaction. Specifically, the transgenic animals contain at least one transgene capable of expressing a target pre-mRNA molecule. The invention provides methods, based on utilization of the transgenic animals, for assessing the specificity and efficiency of a pre-trans-splicing molecule (PTM) designed to interact with a target pre-mRNA and mediate a trans-splicing reaction resulting in the generation of a novel chimeric RNA molecule. The present invention further relates to the transgenic expression of PTM molecules in animals to determine gene function, i.e, functional genetics. The present invention is based on the successful generation of a transgenic animal expressing a target pre-mRNA and, moreover, the use of that animal to detect accurate in vivo trans-splicing reactions in the presence of a PTM.

### 2. BACKGROUND OF THE INVENTION

DNA sequences in the chromosome are transcribed into pre-mRNAs which contain coding regions (exons) and generally also contain intervening non-coding regions (introns). Introns are removed from pre-mRNAs in a precise process called splicing (Chow et al., 1977, Cell 12:1-8; and Berget, S. M. et al, 1977, Proc. Natl. Acad. Sci. USA 74:3171-3175). Splicing takes place as a coordinated interaction of several small nuclear ribonucleoprotein particles (snRNP's) and many protein factors that assemble to form an enzymatic complex known as the spliceosome (Moore et al, 1993, in The RNA World, R. F. Gestland and J. F. Atkins eds. (Cold Spring Harbor Laboratory Press, Cold Spnng Harbor, N.Y.); Kramer, 1996, Annu. Rev. Biochem., 65:367-404; Staley and Guthrie, 1998, Cell 92:315-326).

Pre-mRNA splicing proceeds by a two-step mechanism. In the first step, the 5′ splice site is cleaved, resulting in a “free” 5′ exon and a lanat intermediate (Moore, M. J. and P. A. Sharp, 1993, Nature 365:364-368). In the second step, the 5′ exon is ligated to the 3′ exon with release of the intron as the lariat product. These steps are catalyzed in a complex of small nuclear nbonucleoproteins and proteins called the spliceosome.

In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Nat'l. Acad. Scl. USA, 87:8879; Davis et al., 1995, J. Biol. Chem. 270:21813) and in plant mitochondna (Malek et al., 1997, Proc. Nat'l. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cissplicing. In addition, sequences at the 3′ splice site and some of the snRNPs which catalyze the trans-splicing reaction, closely resemble their counterparts involved in cis-splicing.

Trans-splicing may also refer to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Nat'l. Acad. Sci. USA 86•8020). In addition, trans-splicing of c-myb pre-RNA has been demonstrated (Vellard, M. et al Proc. Nat'l. Acad. Sci., 1992 89:2511-2515) and more recently, RNA transcripts from cloned SV40 trans-spliced to each other were detected in cultured cells and nuclear extracts (Eul et al, 1995, EMBO. J. 14:3226). However, naturally occurring trans-splicing of mammalian pre-mRNAs is thought to be an exceedingly rare event.

In vitro trans-splicing has been used as a model system to examine the mechanism of splicing by several groups (Konarska & Sharp, 1985, Cell 46:165-171 Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base paring was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Nat'l. Acad. Sci. USA 92:7056-7059). These reactions occur at relatively low frequencies and require specialized elements, such as a downstream 5′ splice site or exonic splicing enhancers.

Until recently, the practical application of targeted trans-splicing to modify specific target genes has been limited to catalytic RNA such as group I ribozyme-based mechanisms. Using the Tetrahymena group I ribozyme, targeted trans-splicing was demonstrated in E. coli. (Sullenger B A. and Cech. T. R., 1994, Nature 341:619-622), in mouse fibroblasts (Jones, J. T. et al., 1996, Nature Medicine 2:643-648), human fibroblasts (Phylacton, L. A. et al. Nature Genetics 18:378-381) and human erythroid precursors (Lan et al., 1998, Science 280:1593-1596).

While many applications of targeted RNA trans-splicing driven by modified group I ribozymes have been explored, it is only recently that targeted trans-splicing mediated by native mammalian splicing machinery, i.e., spliceosomes, has been reported. For example, spliceosome-mediated RNA trans-splicing has been effective in correcting the delta F508 CFTR mutant protein in vitro using human cystic fibrosis (CF) polarized airway epithelial cells and in human CF bronchial xenografts (Liu et al, 2002, Nature Biotechnology, 20:47-52). In addition, compositions and methods of using pre-trans-splicing molecules have been described in U.S. Pat. Nos. 6,013,487, 6,083,702, 6,280,978, and in co-pending U.S. patent application Ser. Nos. 09/756,095, 09/756,096, 09/756,097 the disclsoures of which are incorporated by referencin their entireties. These references demonstrate successful trans-splicing reactions mediated by PTMs resulting in the formation of a novel chimeric RNA. The resulting chimeric RNA is designed to provide a desired function, or produce a gene product in the specific cell type.

The formation of chimeric RNA molecules via directed trans-splicing reactions will have a variety of different uses including gene regulation, gene repair and suicide gene therapy for treatment of proliferative disorders such as cancer or treatment of genetic, autoimmune or infectious diseases. Transgenic animals, especially mice, have proven to be very useful for studying the effects of vanous treatments on the progression or amelioration of the disease phenotype. The therapeutic application of trans-splicing to treatment of disease necessitates in vivo model systems for testing the efficiency and specificity of trans-splicing reactions. The present invention provides a transgenic animal model system that can be utilized to evaluate the in vivo efficiency of trans-splicing.

### 3. SUMMARY OF THE INVENTION

The present invention relates to the development of an animal model system for in vivo testing of spliceosome-mediated RNA trans-splicing reactions. The present invention provides transgenic animals that have been genetically engineered to contain a transgene capable of expressing a target pre-mRNA that serves as a substrate for trans-splicing reactions mediated by pre-trans-splicing molecules (PTMs). Such PTMs are designed to interact with a target pre-mRNA and efficiently mediate a trans-splicing reaction resulting in the generation of a novel chimeric RNA molecule. The present invention is based on the successful generation of a transgenic animal expressing a target pre-mRNA that was able to serve as a substrate for a trans-splicing reaction indicating that trans-splicing can be used to manipulate the mammalian genome using a transgenic approach.

The invention further provides methods for evaluating the specificity and efficiency of the trans-splicing process using the transgenic animals of the invention. The methods of the invention comprise delivery of a PTM to cells of the transgenic animal wherein the PTM interacts with the target pre-mRNA molecule expressed by the transgenic animal resulting in a spliceosomal mediated trans-splicing reaction that leads to the generation of a novel chimeric RNA molecule. Detection of trans-splicing may be accomplished using a variety of different methods, including but not limited to detection of the chimeric RNA, or detection of the protein or function encoded by the chimeric RNA. In a specific embodiment of the invention, the transgenic animal may be engineered to express a reporter molecule when the PTM is accurately trans-spliced into the target pre-mRNA. The reporter molecule may be detected externally in the animal using a variety of different means. The invention further provides for the use of targeted trans-splicing in transgenic animals to modify gene function thereby providing a system for studying functional genomics at the cellular level. Specifically, the system may be used to target expression of chimeric mRNAs, resulting from trans-splicing, to a cell expressing a specific target pre-mRNA.

### 4. DESCRIPTION OF THE FIGURES

FIG. 1 is a schematic diagram of a trans-splicing reaction that generates a chimeric RNA capable of encoding functional β-galactosidase activity.

FIG. 2 shows a schematic diagram of a defective (non-functional) LacZ mini-gene pre-mRNA target containing the human ubiquitin intron and a second target intron, CFTR intron 9.

FIG. 3 shows a schematic diagram of a defective (non-functional) LacZ mini-gene pre-mRNA target without the human ubiquitin intron.

FIG. 4 shows a schematic diagram of pAd-LacZ PTM24 used to express PTM 24 in the transgenic animal.

FIG. 5 shows genomic southern blots of pCUBT44.2 transgenic mice. Genomic DNA was harvested from six founder lines and several F1 and F2 offspring in an effort to determine the number of integration sites for each of the transgenic lines. Genomic DNA was digested with BamHI and Southern blots were probed with a ³²P-labeled LacZ probe. Of the four lines that went germ line (18009/2, 17907/2, 18005/3, 18154/4) all but one (18009/2) appears to have a single integration site. Founder line 18009/1 has not yet gone germ line and Founder 17858/1 which tested transgene positive by PCR and negative by Southern blot has also not gone germ line.

FIG. 6 shows the reconstitution of β-galactosidase gene expression in transgenic mice using pCUBT4.2 target (LacZ mutant mini-gene) and recombinant adenovirus encoding LacZ PTM24. Panels A, C and E are in situ histochemically stained muscle samples, while Panels B, D, F-H are frozen sections (6 μm) from the same muscle samples. Left of each panel labels that mouse lines and right of each panel labels the adenoviral vector used for in vivo infection. Arrows point to β-galactosidase expressing myofils in the pCUBT4.2 transgenic line.

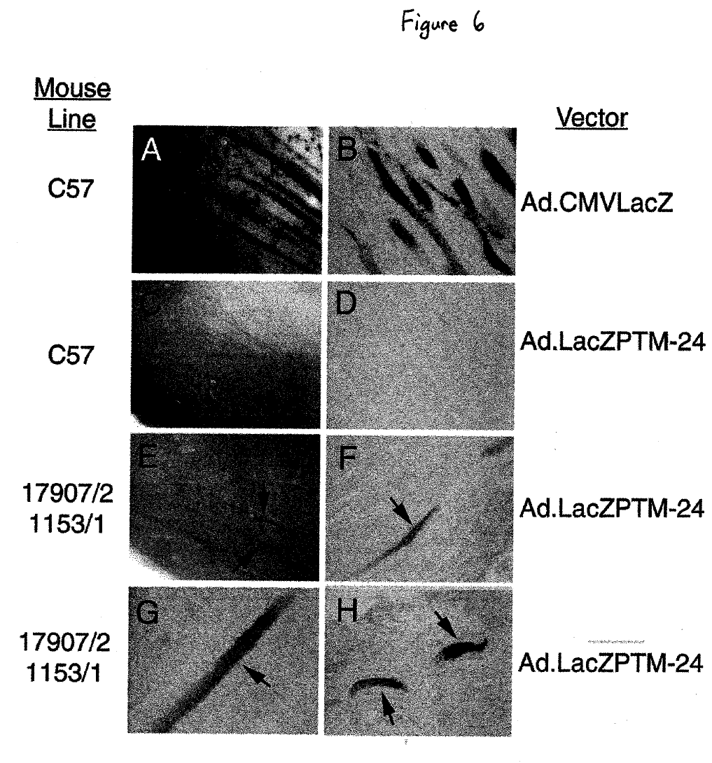

### 5. DETAILED DESCRIPTION OF THE INVENTION

The present invention relates to an in vivo animal model system for in vivo testing of spliceosome mediated RNA trans-splicing reactions. The present invention provides transgenic animals that express a target pre-mRNA molecule which is the substrate for a trans-splicing reaction The transgenic animal of the present invention can be used for evaluation of the efficiency and specificity of trans-splicing reactions in vivo.

### 5.1. Structure of the Transgene

The present invention provides for transgenic animals, and methods for generating such animals, that have been genetically engineered to contain a transgene capable of expressing a target pre-mRNA molecule that serves as a substrate for a spliceosomal mediated trans-splicing reaction.

The structure of the transgene to be used in the generation of the transgenic animals of the invention will depend upon the structure of the PTM molecule to be tested. In general, PTMs comprise (i) one or more target binding domains that target binding of a specific PTM to a target pre-mRNA (ii) a 3′ splice region that includes a branch point, pyrimidine tract and a 3′ splice acceptor site and/or 5′ splice donor site; and (in) one or more spacer regions that separate the RNA splice site from the target binding domain. Additionally, the PTMs can be engineered to contain any nucleotide sequence encoding a translatable protein product or nucleolide sequence that inhibits the translation of the chimenc RNA molecule.

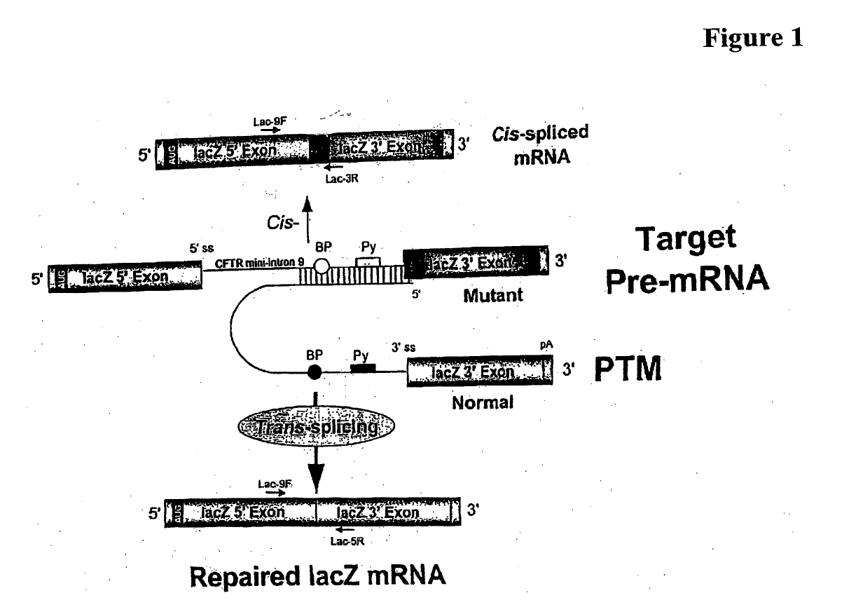

In an embodiment of the present invention, the PTMs to be tested are engineered to express a portion of a reporter molecule. In such instances, an accurate trans-splicng reaction between the PTM and the target pre-mRNA will result in the formation of a chimeric RNA capable of encoding a reporter molecule (FIG. 1).

Transgenes to be used for generating the transgenic animals of the invention will be capable of encoding a target pre-mRNA comprising (i) binding domains that are complementary to and in anti-sense orientation to the specific PTM to be tested; and (ii) at least one intron sequence, which is targeted for removal by trans-splicing, flanked by exon sequences or at least one consensus splice site (or a sequence that functions as a consensus splice site). The target pre-mRNA intron/exon sequences are designed to contain all the necessary consensus sequences required for a splicesomal mediated splicing reaction. Such sequences include a 3′ splice region that includes a branch point, a 3′ splice acceptor AG site and/or a 5′ splice donor site. The 3′ splice site may additionally contain a pyrimidne tract. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and/=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine). The underlined A is the site of branch formation. A polypyrimidine tract is located between the branch point and the splice site acceptor and is important for different branch point utilization and 3′ splice site recognition. In addition, any modified consensus sequences that maintain the ability to function as 5′ donor splice sites and 3′ splice regions may be used in the practice of the invention.

The binding domain of the target pre-mRNA may contain multiple binding domains which are complementary to and in anti-sense orientation to the target binding domain region of the specific PTM to be tested. As used herein, a binding domain is defined as any sequence that confers specificity of binding and anchors the target pre-mRNA closely in space to the PTM so that the spliceosome processing machinery of the nucleus can trans-splice a portion of the PTM to a portion of the target pre-mRNA. The binding domains may comprise up to several thousand nucleotides. In a preferred embodiment of the invention the binding domains may comprise at least 10 to 30 and up to several hundred nucleotides. The specificity of the target pre-mRNA for a PTM can be increased significantly by increasing the length of the binding domain. For example, the binding domain may comprise several hundred nucleotides or more. In addition, although the binding domain may be “linear” it is understood that the RNA may fold to form secondary structures that may stabilize the complex thereby increasing the efficiency of splicing. Absolute complementarity, although preferred, is not required. A sequence “complementary” to a portion of the PTM, as referred to herein, means a sequence having sufficient complementarity to be able to hybridize with the PTM, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with a PTM it may contain and still form a stable duplex. One skilled in the art can ascertain a tolerable degree of mismatch or length of duplex by use of standard procedures to determine the stability of the hybridized complex.

In a preferred embodiment of the invention, to facilitate detection of the trans-splicing reaction, the exon sequences of the target pre-mRNA may comprise sequences encoding a translatable protein capable of producing a reporter molecule. The target pre-mRNA is engineered in such a way that trans-splicing between the target pre-mRNA and the PTM results in the formation of a chimeric RNA capable of encoding a reporter molecule. Such reporter molecules include, but are not limited to, bioluminescent and fluorescent molecules, receptors, ion channel components, enzymes, and protein/peptide tags (Yu et al., 2000 Nature Medicine 6:933-937; MacLarent et al., 2000 Biol Psychiatry 48:337-348; Zaret et al., 2001 J. Nuclear Cardiology March/April 256-266; Ray et al., 2001 Seminars in Nuclear Medicine 31:312-320; Lok, 2001 Nature 412:372-374; Allport et al., 2001 Experimental Hematology 29:1237-1246; Berger and Gambhir, 2000 Breast Cancer Research 3:28-35; Cherry and Gambhir, 2001, ILAR Journal 42:219-232). Bioluminescent molecules include but are not limited to firefly, Renilla or bacterial luciferase. Fluorescent molecules include, for example, green fluorescent protein or red fluorescent protein.

In yet another embodiment of the invention, the reporter molecule may be an enzyme such as β-galactosidase (Louie et al., 2000 Nature Biotechnology 15:321-325), cytosine deaminase, herpes simplex virus type I thymidine kinase, creatine kinase (Yaghoubi et al., 2001 Human Imaging of Gene Expression 42:1225-1234; Yaghoubi et al., 2001 Gene Therapy 8:10721080; Iyer et al., 2001 J. Nuclear Medicine 42:96-105), or arginine kinase, to name a few.

Alternatively, the nucleotide sequences can encode for an intracellular and/or extracellular marker protein, such as a receptor, which is capable of binding to a labeled tracer that has a binding affinity for the expressed marker protein. Such proteins include, for example, the dopamine 2 receptor, somatostatin receptor, oxotechnetate-binding fusion proteins, gastnnreleasing peptide receptor, cathepsin D, the transferrin receptor or the CFTR C1 ion channel.

Nucleotide sequences encoding peptide tags, also referred to as epitope tags, may also be included in the structure of the target pre-mRNA. In a preferred embodiment of the invention, the epitope is one that is recognized by a specific antibody or binds to a specific ligand, each of which may be labeled, thereby providing a method for imaging of cells expressing the accurately spliced chimeric RNAs. Epitopes that may be used include, but are not limited to, AU1, AU5, BTag, c-myc, FLAG, Glu-Glu, HA, His6, HSV, HTTPHH, IRS, KT3, Protien C, S-Tag, T7, V5, or VSV-G.

Cloning techniques well known to those of skill in the art may be used for cloning a nucleic acid molecule encoding a target pre-mRNA into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY. The nucleic acid molecule encoding the target pre-mRNA of interest may be recombinantly engineered into a variety of host vector systems that also provide for replication of the DNA in large scale and contain the necessary elements for directing the transcription of the target pre-mRNA. The use of such a construct to generate a transgenic animal, will result in animals where target pre-mRNA is transcribed in sufficient amounts to form complementary base pairs with the PTM to be tested and thereby facilitate a trans-splicing reaction between the complexed nucleic acid molecules.

Vectors encoding the target pre-mRNA of interest, can be plasmid, viral, or others known in the art, for replication and expression in mammalian cells. Expression of the sequence encoding the target pre-mRNA can be regulated by any promoter known in the art to act in mammalian cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Scl. U.S.A. 78:1441-1445), the regulatory sequences of the metallothionein gene (Brinster et al., 1982, Nature 296:39-42), the viral CMV promoter, the human chononic gonadotropin-β promoter (Hollenberg et al., 1994, Mol, Cell. Endocrinology 106:111-119), etc. Any type of plasmid, cosmid, or viral vector can be used to prepare the recombinant DNA construct which can then be used to generate the transgenic animals of the invention.

### 5.3. Generation of Transgenic Animals

The present invention provides for genetically engineered non-human animals that express a target pre-mRNA nucleic acid molecule. Recombinant nucleic acid molecules capable of encoding a target pre-mRNA molecule, i e., a transgene, may be introduced into the genome of non-human animals using any of the known methods for generating transgenic animals. The term “transgenic animals” refers to non-human animals which have incorporated a foreign gene into their genome. Transgenic mice have been generated using a variety of different methods, including those utilized to produce mice expressing globin (Wagner et al., 1981, Proc. Natl. Acad. Sci. U.S.A. 78.6376-6380), transfemn (McKnight et al., 1983, Cell 34:335-341), immunoglobulin (Brinster et al, 1983, Nature 306:332-336; Ritchie et al., 1984, Nature 312:517-520; Goodhardt et al., 1987, Proc. Natl. Acad. Sci. U.S.A. 84:4229-4233; Stall et al., 1988, Proc. Natl. Acad. Sci. U.S.A. 85:3546-3550), human major histocompatibility complex class I heavy and light chain (Chamberlain et al., 1988, Proc. Natl. Acad. Sci. U.S.A. 85:76907694), functional human interleukin-2 receptors (Nishi et al., 1988, Nature 331:267-269), rat myosin light-chain 2 (Shani, 1985, Nature 314:283-286), and hepatitis B virus (Chisari et al., 1985, Science 230:1157-1163) genes.

Methods for generating such genetically engineered animals are known in the art and include, for example, pronuclear microinjection, retroviral mediated gene transfer into germ line cells or embryos, blastomere-embryo aggregation, gene targeting in embryonic stem cells, electroporation of embryos, nuclear transplantation and spermatozoa-mediated transfer. Methods for generating genetically engineered animals are reviewed, for example, by Pinkirt et al (1995, Transgenic Animal Modeling, in Molecular Biology and Biotechnology, Myers, ed. pp.90-107) and numerous biology manuals including, for example, Hogan et al. (1994, Manipulating the Mouse Embryo: A Laboratory Manual, 2nd Edition), the disclosure of which is incorporated herein by reference.

In an embodiment of the invention, male and female mice from a defined inbred genetic background are mated. Twelve hours later, the female is sacrificed and the fertilized—eggs are removed from the uterine tubes and recombinant DNA encoding the target pre-mRNA molecule is then microinjected (100-1000 molecules per egg) into the pronucleus of the fertilized egg. The fertilized egg is then implanted into a pseudo-pregnant female mouse (previously mated with a vasectomized male) where the embryo develops for the full gestation penod of 20-21 days.

Alternatively, embryonic stem cells may be used. Recombinant DNA encoding the target pre-mRNA molecule is microinjected into the embryonic stem cell. The embryonic stem cell is then injected into a blastocyst, which is then implanted into a pseudo-pregnant female mouse.

Once delivered, the pups are weaned from the mother and tested for the presence of foreign DNA. In a specific embodiment, a portion of the tail (a dispensable organ) is removed and DNA extracted. DNA-DNA hybridization (in a dot blot, slot blot or Southern blot test) may be employed to determine whether the mice carry the transgene of the invention and the copy number of the transgene. In addition, polymerase chain reaction may be utilized to detect the presence and the copy number of the transgene. The transgenic mice may be bred to pass along the foreign gene in a normal (Mendelian) fashion. Thus, mating two homozygous mice with the transgenic DNA will result in the offspring carrying two copies of the transgene.

The present invention is not limited to any one species of animal, but provides for any non-human animal species which may be appropriate. For example, mice, guinea pigs, rabbits and pigs, sheep, cows, goats, and horses, to name but a few, may provide useful transgenic systems.

### 5.4. Use of Transgenic Animals

PTM molecules are designed to interact with a target pre-mRNA molecule and mediate a trans-splicing reaction resulting in the generation of a novel chimeric RNA molecule. The novel chimeric RNA resulting from the trans-splicing reaction may itself perform a function such as inhibiting the translation of RNA, or alternatively, the chimeric RNA may encode a protein that complements a defective or inactive protein in the cell, encodes a toxin which kills the specific cells or provides new functions such as recombinase activity or modulation of transcription. PTMs can be used for the treatment of various diseases including, but not limited to, genetic, infectious or autoimmune diseases and proliferative disorders such as cancer.

A transgenic animal, expressing a specific target pre-mRNA, provides a useful animal model system for assessing the specificity and efficiency of PTM mediated in vivo trans-splicing reactions. Such animals may be to identify PTM molecules capable of efficiently mediating a specific trans-splicing reaction.

The present invention relates to methods for evaluating the ability of a PTM molecule to mediate a trans-splicing reaction. A trans-splicing reaction between the target pre-mRNA and the PTM will result in the formation of a novel chimeric RNA molecule. The methods of the invention rely on the use of transgenic animals engineered to express a target pre-mRNA that will serve as a substrate for the particular PTM being tested. Methods for assaying PTMs comprise the following steps; (i) delivery of the PTM to cells of the transgenic animal, wherein the PTM will interact with the expressed target pre-mRNA, and mediate a trans-splicing reaction resulting in formation of a novel chimeric RNA molecule; and (ii) assaying for the presence or function of the novel chimeric RNA molecule.

Various delivery systems are known and can be used to transfer the PTMs to be tested into cells of the transgenic animal, e g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral or other vector, injection of DNA, electroporation, calcium phosphate mediated transfection, etc.

In an embodiment, nucleic acids comprising a PTM, or sequences encoding a PTM, are administered to a transgenic animal to assess PTM function, by way of gene delivery into a transgenic cell expressing the target pre-mRNA. In this embodiment of the invention, the nucleic acid mediates an effect by promoting PTM production. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev Biochem. 62:191-217; 1993, TIBTECH 11(5):155-215.

In a preferred embodiment, the PTM, or a nucleic acid encoding a PTM, is directly administered in vivo to the transgenic animal. This can be accomplished by any of numerous methods known in the art, e g., by constructing it as part of an appropriate nucleic acid expression vector and administenng it so that it becomes intracellular, e g. by infection using a defective or attenuated retroviral or other viral vector (see U.S. Pat. No. 4,980,286), or by direct injection of naked DNA, or by use of microparticle bombardment (e.g, a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e g, Wu and Wu, 1987, J. Biol. Chem. 262:4429-4432).

In an embodiment of the invention, a viral vector that encodes the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors engineered to express PTMs can be used for gene delivery to cells or tissues of the transgenic animal. (See, Kozarsky and Wilson, 1993, Current Opinion in Genetics and Development 3:499-503 for a review of adenovirus-based gene delivery).

The presence of chimeric RNA, or activity of the protein encoded for by the chimeric mRNA, resulting from the PTM mediated trans-splicing reaction can be readily detected, e.g, by obtaining an animal tissue sample (e g., from biopsy tissue) and assaying it in vitro for chimeric mRNA or protein levels or detection of the reporter molecule. Many methods standard in the art can be thus employed, including but not limited to immunoassays to detect and/or visualize the protein encoded for by the chimeric mRNA (e.g., Western blot, immunoprecipitation followed by sodium dodecyl sulfate polyacrylamide gel electrophoresis, immunocytochemistry, etc.) and/or hybridization assays to detect formation of chimeric mRNA by detecting and/or visualizing the presence of chimeric mRNA, e.g. Northern assays, dot blots, in situ hybridization, and Reverse-Transcription PCR, etc.

In vitro and/or in vivo methods may be utilized to detect expression of a reporter molecule resulting from formation of a chimeric RNA. In vitro assays for detection of reporter molecule expression and/or function, include but are not limited to, in situ hybridization assays, detection of fluorescent or bioluminescent signals or enzymatic assays. For, in vivo detection of reporter molecule expression, cells can be imaged using a number of methods well known to those of skill in the art. Such methods include, for example, use of a CCD low-light monitoring system, positron emission tomography (PET), single photon emission computed tomography (SPECT), magnetic resonance imaging (MRI), ultrasound (US), and endoscopic optical coherence tomography. In instances where the reporter molecule does not provide a label for imaging, a tracer molecule is added to detect expression of the reporter molecule. The tracer molecule can be labeled in a variety of different ways, including but not limited to, fluorescent, bioluminescent and radioactive labeling. The tracer is designed to bind to the reporter molecule thereby providing a signal for cells expressing the novel chimeric RNA.

### 5.5. Transgenic Animals Expressing PTMS

The present invention further relates to the generation of transgenic animals capable of expressing PTM molecules. Such animals can be used to study gene function, ie., functional genetics. In an embodiment of the invention, the PTMs are engineered to target trans-splicing between a pre-mRNA molecule expressed within a specific cell type and the PTM. For example, a PTM capable of expressing a cytotoxic molecule may be used for targeted cell ablation. Such cytotoxic molecules include but are not limited to diptheria toxin or thymidine kinase (Yagi et al., 1993, Annl Biochem 214:77-86; Robinson et al., 1995, Hum Gene Ther 6:137-43; Harrison et al., 1992, AIDS Res Hum Retrovirus 8:39-45; Dinges et al., 1995 Hum Gene Ther 6:1437-45). PTMs may also be engineered to encode functional recombinase activity such as CRE or Flip to target genome manipulation in a cell expressing a specific target pre-mRNA. For example, PTM-CRE directed recombination can be used to specifically activate or inactivate gene expression from a second floxed loci containing the necessary recombinational sequences flanking a transgene. In such an instance, recombination at a second locus can be regenerated by expression of the target pre-mRNA. Additionally, PTMs for use in the generation of transgenic animals can be designed to express specific activators and/or repressors of transcription. Such activators/repressors may be utilized to modulate the expression of a second transgenic locus. For example, tet or estrogen receptor regulated gene operon components can be used to target activation or inhibition of tet or estrogen regulated genes at a second locus in the transgenic animal.

### 6. EXAMPLE

The following example, demonstrates the successful generation of a transgenic animal that expresses a target pre-mRNA. Moreover, contacting cells of the transgenic mouse with a PTM resulted in an accurate in vivo trans-splicing reaction.

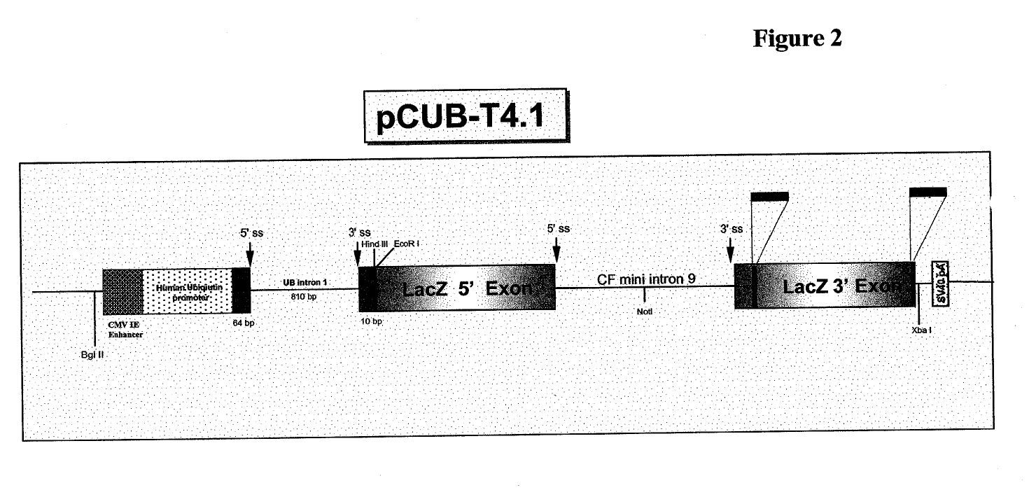

Using the construct depicted in FIG. 2B, transgenic mice were generated. Plasmid DNA was linearized and a DNA fragment comprising the lacZ minigene was isolated from the bacterial backbone. Transgene DNA was injected into the pro-nucleus of a single cell mouse embryos and implanted into pseudopregannt females at 2-8 cell stage. Offspring were screened for the incorporation of transgene nucleic acid sequences in the genome by polymerase chain reaction and postitive founders were bred against C57 mice to determine the extent to which the transgene was transmitted to the germ line. F1 transgene positive animals were then bred against C57 mice and the F2 generation of positive animals were screened for the ezpression of the encoded PTM target.

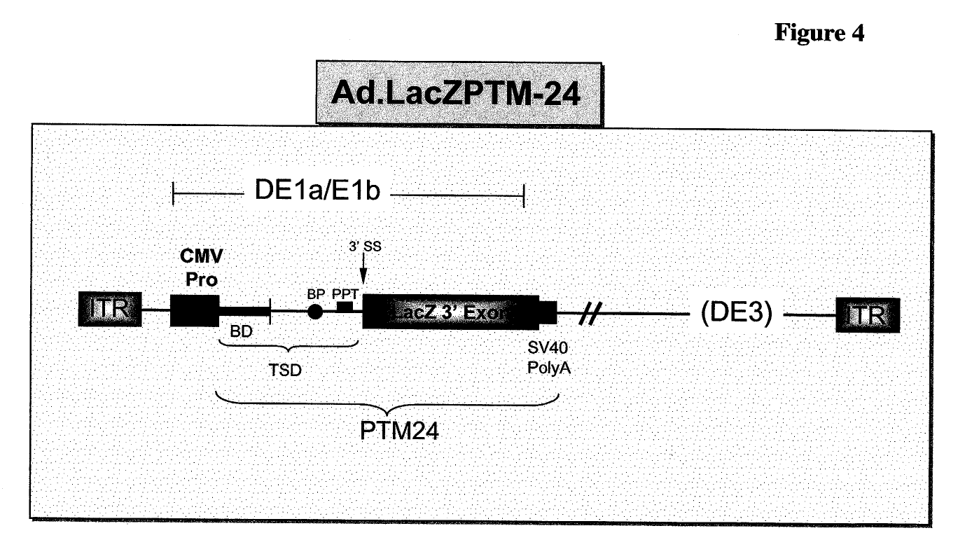

Genomic DNA was harvested from six founder lines and several F1 and F2 offspring in an effort to determine the number of integration sites for each of the transgenic lines. Genomic DNA was digested with BamHI and Southern blots were probed with a ³²P-labled LacZ probe (FIG. 4). Of the four lines that went germ line (18009/2, 17907/2, 18005/3, 18154/4) all but one (18009/2) appears to have a single integration site. Founder line 18009/1 has not yet gone germ line and Founder 17858/1 which tested transgene positive by PCR and negative by Southern blot has also not gone germ line.

The transgenic mice were then assayed to determine whether in vivo trans-splicing reactions could occur within the cells of the transgenic animals. Reconstitution of β-galactosidase gene expression in pCUBT4.2 (LacZ mutant mini-gene) transgenic mice was tested using a recombinant adenovirus encoding LacZPTM-24. The recombinant adenovirus was generated by cloning the LacZPTM-24 transgene into the adenoviral shuttle plasmid pAd.CMVlink followed by generation of recombinant adenovirus (see, Duan et al., Current Protocols in Human Genetics, 1998, John Wiley & Sons, Inc.). For a detailed description of the LacZPTM24 construct see patent application Ser. No. 09/941,492 the disclosure of which is incorporated herein by reference in its entirety.

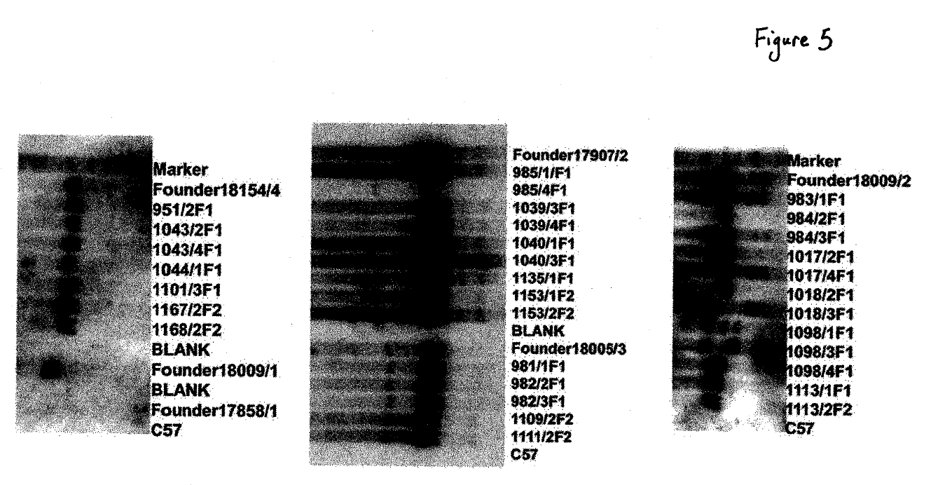

pCUBT4.2 transgenic mice and Ad.LacZPTM-24 virus were used to test for in vivo trans-splicing mediated correction of a mutated LacZ-minigene in skeletal muscle. Approximately 2×10¹⁰ particles of Ad.LacZPTM-24 virus were injected into one tibialis muscle of C57 control mice (FIG. 5, Panels C and D) or an F2 mouse (1153/1) from the pCUBT4.2 17907/2 Founder line (FIG. 5, Panels E-H). Additionally, as a positive control, the tibialis anterior muscle from C57 mice was also infected with 2×1010 particles of Ad.CMVLacZ that expresses the full-length and functional β-galactosidase gene (FIG. 5, Panels A and B). Muscle samples were harvested at 5 days post-infection and stained for functional β-galactosidase protein using X-gal histochemistry. Panels A, C, and E are en face histochemically stained muscle samples, while Panels B, D, F-H are frozen sections (6 um) from the same muscle samples. Left of each panel labels the mouse line, and right of each panel labels the adenoviral vector used for in vivo infection. Arrows point to β-galactosidase expressing myofibers in the pCUBT4.2 transgenic line. As demonstrated in FIG. 5, β-galactosidase was successfully reconstituted demonstrating successful and accurate in vivo trans-splicing.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties

We claim:

1. A transgenic non-human animal whose germ cells and somatic cells contain an exogenous nucleic acid molecule which is transcribed to produce a target pre-mRNA molecule comprising:
i) one or more target binding domains that target binding of the target pre-mRNA molecule to a pre-trans-splicing molecule; and
(ii) at least one intron sequence or consensus splice site,
wherein said target pre-mRNA molecule is a substrate for a trans-splicing reaction in the presence of the pre-trans-splicing molecule.

2. A transgenic mouse whose germ cells and somatic cells contain a exogenous nucleic acid molecule which is transcribed to produce a target pre-mRNA molecule comprising:
(i) one or more target binding domains that target binding of the target pre-mRNA molecule to a pre-trans-splicing molecule; and
(it) at least one intron sequence or consensus splice site
wherein said target pre-mRNA molecule is a substrate for a trans-splicing reaction in the presence of the pre-trans-splicing molecule.

3. The transgenic animal of claim 1, wherein the target pre-mRNA further comprises nucleotide sequences encoding a reporter molecule.

4. The transgenic mouse of claim 2, wherein the target pre-mRNA further comprises nucleotide sequences encoding a reporter molecule.

5. The transgenic animal of claim 1, wherein the target pre-mRNA contains a 3′ or 5′ consensus sequences required for spliceosomal mediated splicing.

6. The transgenic mouse of claim 2, wherein the target pre-mRNA contains 3′ or 5′ consensus sequences required for spliceosomal mediated splicing.

7. The transgenic animal of claim 1, further comprising a exogenous nucleic acid molecule encoding a pre-trans-splicing molecule.

8. The transgenic mouse of claim 2, further comprising a exogenous nucleic acid molecule encoding a pre-trans-splicing molecule.

9. The transgenic animal of claim 7 wherein the exogenous nucleic acid molecule is an expression vector.

10. The transgenic mouse of claim 8 wherein the exogenous nucleic acid molecule is an expression vector.

11. The transgenic animal of claim 7 wherein the expression vector is a viral vector.

12. The transgenic mouse of claim 8 wherein the expression vector is a viral vector.

13. The transgenic animal of claim 11 wherein the viral vector is an adenovirus vector.

14. The transgenic mouse of claim 12 wherein the viral vector is an adenovirus vector.

15. The transgeneic animal of claim 3 wherein the reporter molecule is an enzyme.

16. The transgenic mouse of claim 4 wherein the reporter molecule is an enzyme.

17. The transgenic animal of claim 3 wherein the reporter molecule is a bioluminescent or chemoluminescent molecule.

18. The transgenic mouse of claim 4 wherein the reporter molecule is a bioluminescent or chemoluminescent molecule.

19. The transgenic animal of claim 15 wherein the enzyme is β-galactosidase.

20. The transgenic mouse of claim 16 wherein the enzyme is β-galactosidase.

21. The transgenic animal of claim 1 wherein the target pre-mRNA molecule is encoded by pCUBT4.2.

22. The transgenic mouse of claim 2 wherein the target pre-mRNA molecule is encoded by pCUBT4.2.

23. A method of producing the transgenic animal of claim 1, wherein said method comprises:
i) introducing an exogenous nucleic acid molecule into an embryonic stem cell, wherein said nucleic acid molecule is transcribed to form a target pre-mRNA molecule comprising:
(a) one or more target binding domains that target binding of the target pre-mRNA molecule to a pre-trans-splicing molecule; and
(b) at least one intron sequence or consensus splice site
wherein said target pre-mRNA molecule is a substrate for a trans-splicing reaction in the presence of a pre-trans-splicing molecule;
ii) injecting the embryonic stem cell into a blastocyst,
iii) transplanting said blastocyst into the reproductive tract of an animal,
iv) allowing said blastocyst to develop into an animal whose genome contains said exogenous nucleic acid molecule, and
v) screening said animal of step (iv) to identify a transgenic animal whose genome comprises said selectable marker.

24. A method of producing the mouse of claim 2, wherein said method comprises:
i) introducing an exogenous nucleic acid molecule into an embryonic stem cell, wherein said nucleic acid molecule is transcribed to form a target pre-mRNA molecule comprising:
(a) one or more target binding domains that target binding of the target pre-mRNA molecule to a pre-trans-splicing molecule; and
(b) at least one intron sequence or consensus splice site, and
wherein said target pre-mRNA molecule is a substrate for a trans-splicing reaction in the presence of the pre-trans-splicing molecule;
ii) injecting the embryonic stem cell into a blastocyst,
iii) transplanting said blastocyst into the reproductive tract of a mouse,
iv) allowing said blastocyst to develop into a mouse whose genome contains said exogenous DNA construct, and
v) screening said mouse of step (iv) to identify a transgenic mouse whose genome comprises said selectable marker.

25. A method of producing the transgenic animal of claim 1, wherein said method comprises:
i) introducing an exogenous nucleic acid molecule into a fertilized egg, wherein said nucleic acid molecule is transcribed to form a target pre-mRNA molecule comprising:
(a) one or more target binding domains that target binding of the target pre-mRNA molecule to a pre-trans-splicing molecule; and
(b) at least one intron sequence or consensus splice site, and
wherein said target pre-mRNA molecule is a substrate for a trans-splicing reaction in the presence of the pre-trans-splicing molecule;
ii) transplanting said fertilized egg into the animal,
iii) allowing said fertilized egg to develop into an animal whose genome contains said exogenous nucleic acid molecule, and
iv) screening said animal of step (iii) to identify a transgenic animal whose genome comprises said exogenous nucleic acid.

26. A method of producing the transgenic mouse of claim 2, wherein said method comprises:
i) introducing an exogenous nucleic acid molecule into a fertilized egg, wherein said nucleic acid molecule is transcribed to form a target pre-mRNA molecule comprising:
(a) one or more target binding domains that target binding of the target pre-mRNA molecule to a pre-trans-splicing molecule; and
(b) at least one intron sequence or consensus splice site, and wherein said target pre-mRNA molecule is a substrate for a trans-splicing reaction in the presence of the pre-trans-splicing molecule;
ii) transplanting said fertilized egg into a mouse,
iii) allowing said fertilized egg to develop into mouse whose genome contains said exogenous nucleic acid molecule, and
iv) screening said mouse of step (iii) to identify a transgenic mouse whose genome comprises said exogenous nucleic acid.

27. The method of claim 23, 24, 25 or 26 wherein the exogenous nucleic acid molecule is pCUBt4.2

28. A method of producing a chimeric RNA molecule in the transgenic animal of claim 1 comprising contacting the target pre-mRNA molecule expressed within the cells of the animal with a pre-trans-splicing molecule recognized by nuclear splicing components wherein said exogenous nucleic acid molecule comprises:
(i) one or more target binding domains that target binding of the nucleic acid molecule to the target pre-mRNA expressed within a cell;
(ii) a 3′ splice region comprising a branchpoint and a 3′ splice acceptor site or a 5′ splice site; and
(iii) nucleotide sequence to be trans-spliced to the target pre-mRNA;
wherein said nucleic acid molecule is recognized by nuclear splicing components within the cell.

29. A method of producing a chimeric RNA molecule in the transgenic mouse of claim 2 comprising contacting the target pre-mRNA molecule expressed within the cells of the animal with a pre-trans-splicing molecule recognized by nuclear splicing components wherein said exogenous nucleic acid molecule comprises:
(i) one or more target binding domains that target binding of the nucleic acid molecule to the target pre-mRNA expressed within a cell;
(ii) a 3′ splice region comprising a branchpoint and a 3′ splice acceptor site or 5′ splice site; and
(iii) nucleotide sequence to be trans-spliced to the target pre-mRNA;
wherein said nucleic acid molecule is recognized by nuclear splicing components within the cell.

30. The method of claim 28 or 29, wherein the pre-trans-splicing molecule is PTM 24.

31. The method of claim 28 or 29 wherein the pre-trans-splicing molecule is encoded by the adenovirus vector Ad LacZPTM-24.

32. The method of claim 28 or 29 wherein said target pre-mRNA comprises a nucleotide sequence encoding a reporter molecule.

33. A method for testing the ability of a pre-trans-splicing molecule to mediate a trans-splicing reaction comprising;
(i) contacting the pre-target mRNA expressed in the transgenic animal of claim 1 with the pre-trans-splicing molecule wherein a portion of the pre-trans-splicing trans-splicing molecule is spliced to a portion of the pre-target mRNA to form a chimeric mRNA; and
(ii) detecting the presence of the chimeric mRNA molecule.

34. A method for testing the ability of a pre-trans-splicing molecule to mediate a trans-splicing reaction comprising;
(i) contacting the pre-target mRNA expressed in the transgenic mouse of claim 2 with the pre-trans-splicing molecule wherein a portion of the pre-trans-splicing molecule is spliced to a portion of the pre-target mRNA to form a chimeric mRNA; and
(ii) detecting the presence of the chimeric mRNA molecule.

35. The method of claim 33 or 34 wherein the pre-trans-splicing molecule is encoded by the adenovirus vector Ad.LacZPTM 24.

36. The method of claim 33 or 34 wherein the chimeric mRNA is detected.

37. The method of claim 33 or 34 wherein a reporter molecule encoded by the chimeric mRNA is detected.

38. The method of claim 33 or 34 wherein the reporter molecule is an enzyme.

39. The claim of claim 33 or 34 wherein the reporter molecule is a chemiluminescent or bioluminescent molecule.

40. A recombinant adenovirus vector capable of expressing PTM 24.

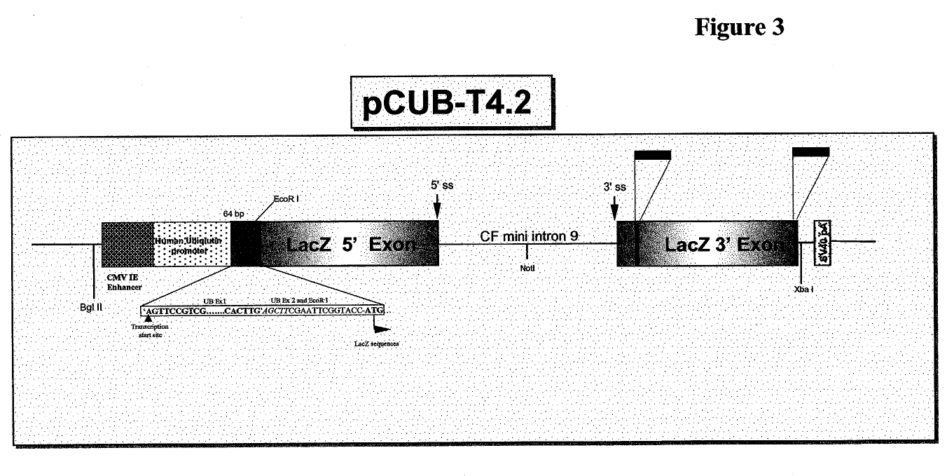

41. The recombinant adenovirus Ad.LacZPTM24 as depicted in FIG. 3.
